(19) United States
(12) Patent Application Publication
(10) Pub. No.: US 2017/0020721 A1
(43) Pub. Date: Jan. 26, 2017

(54) Cooled Insulated Headgear Device

(71) Applicant: Mark S. Kobilca, Lemont, IL (US)

(72) Inventor: Mark S. Kobilca, Lemont, IL (US)

(21) Appl. No.: 15/178,426

(22) Filed: Jun. 9, 2016

Related U.S. Application Data
(60) Provisional application No. 62/173,061, filed on Jun. 9, 2015.

(51) Int. Cl. A61F 7/02; A42B 3/08; A61F 7/10; A42B 1/04; A42B 1/00; A42B 3/04

(52) U.S. Cl. CPC A61F 7/02; A42B 1/006; A42B 3/0406; A61F 7/10; A42B 1/045; A42B 3/08; A61F 2007/0008

(57) ABSTRACT

A headgear device for inhibiting a user from losing hair is provided. The user has a scalp with the hair growing from hair follicles in the scalp. The headgear device comprises a head covering and a cooled gel insert receivable within the head covering. The cooled gel insert cools the scalp and the hair follicles, restricting the flow of blood to the scalp and the hair follicles.

### CLAIM OF PRIORITY

This patent application claims priority under 35 USC 119 (e) (1) from U.S. Provisional Patent Application Ser. No. 62/173,061 filed Jun. 9, 2015, entitled, “Helping Hat,” which is incorporated herein by reference as though the same were set forth in its entirety.

### FIELD OF THE INVENTION

The present invention pertains to the field of headgear, and more specifically to the field of thermally conductive and protective headgear.

### BACKGROUND OF THE INVENTION

Common types of cancer include breast, colon, prostate, and skin cancer as well as various forms of leukemia and lymphoma. After a cancer diagnosis, medical doctors often remove the cancerous growth and recommend a course of treatment that serves to shrink and eradicate remaining cancer cells so a patient might enjoy remission from the disease and a prolonged and happy life. While radiation is implemented to target cancerous growths directly, chemotherapy targets destruction of cancer cells throughout a patient's body.

Chemotherapy typically involves the use of cytotoxic medications that prevent cancer cells from dividing and growing. The chemotherapy success rate depends on the type and stage of cancer being treated, coupled with the patient's overall physical health. Catching cancer early enough and administering chemotherapy to a patient can result in that patient enjoying a lifelong reprieve from their illness. Unfortunately, chemotherapy treatment has negative drawbacks. Debilitating nausea, mouth sores, loss of appetite, chronic fatigue and anemia are painful and irritating side effects caused by chemotherapy. Hair loss is a dreaded side effect of chemotherapy, due to a reasonable concern over one's personal appearance and also because hair loss provides a clear indicator that one is suffering from illness, a fact that many people prefer to keep private.

The prior art has put forth several designs for thermally conductive and protective helmets. Among these are:

US Patent 2012/0210490 to Robert D. Harty describes a head cooling and heating device comprising a webbing adapted to encapsulate the human head. A plurality of cavities defined by said webbing is adapted to reversibly receive a plurality of substrates, wherein the substrates are capable of being cooled or heated. This invention relates to a shock absorbing helmet liner which simultaneously keeps the wearer's head and neck and spine cool or warm. More specifically, this invention relates to a device for regulating body temperature and, in turn, cooling the brain, reducing swelling on the brain, and reducing effects of hyperthermia and fever while simultaneously preventing concussion injuries.

US Patent 2011/0078845 to Cecil D. McKinney describes a thermoregulatory system with a preferred use in combination with headwear, thereby defining a temperature controlled head gear comprising an article of headwear and a thermally conductive pad in conductive association with the thermoregulatory system. The thermoregulatory system in temperature controlled head gear comprises a protective helmet and a pad containing a thermally conductive material, such as a synthetic oil or thermal gel. Examples of thermal gels include gels such as those found in various reusable gel packs or hot and cold packs. Such gels retain a cold or hot temperature, and remain pliable after being subjected to freezing temperatures or microwave radiation in a conventional microwave oven for a sufficient period of time. Other gels are those based on the gelation of xanthan gum, locust bean gum, gum tragacanth, guar gum, hydroxypropyl methylcellulose, absorbent polymers, and the like.

US Patent 2006/0005291 to Nicholas Bedford describes an impact protective item of headwear which has inner and outer impact resistant parts nesting one with respect to the other and shaped to hold between them a replaceable pouch with a gel cooling capability. The article of headwear includes an impact resistant outer part of crown shape and an impact resistant inner part of crown shape of a size and shape adapted to nest with the outer part and at least one intermediate part which is positioned to be intermediate the said outer and inner crown shaped parts. The outer and inner parts are together adapted to support in removable fashion the intermediate part, and where the intermediate part is comprised of a fluid at least in the main portion.

None of these prior art references describe the present invention.

### SUMMARY OF THE INVENTION

It is an object of the present invention to provide an insulated cap filled with a coated gel insert that keeps the interior of the cap icy cold during wear.

The present invention is a headgear device for inhibiting a user from losing hair. The user has a scalp with the hair growing from hair follicles in the scalp. The headgear device comprises a head covering and a cooled gel insert receivable within the head covering. The cooled gel insert cools the scalp and the hair follicles, restricting the flow of blood to the scalp and the hair follicles.

In addition, the present invention is a method for inhibiting a user from losing hair. The user has a scalp with the hair growing from hair follicles in the scalp. The method comprises providing a head covering, positioning a cooled gel insert within the head covering, cooling the scalp and the hair follicles, and restricting the flow of blood to the scalp and the hair follicles.

The present invention further includes a headgear device for inhibiting a user from losing hair. The user has a scalp with the hair growing from hair follicles in the scalp. The headgear device comprises an insulated, hard-sided head covering and a cooled gel insert receivable within the head covering. An electrical cooling system maintains the cooled gel insert at a desired cooled temperature. The insulated head covering inhibits cold air from escaping the helmet during wear. The cooled gel insert cools the scalp and the hair follicles, restricting the flow of blood to the scalp and the hair follicles.

### BRIEF DESCRIPTION OF THE DRAWINGS

FIG. 1 is a perspective view illustrating a cooled gel insert of a cooled insulated headgear device, constructed in accordance with the present invention.

FIG. 2 is a perspective view illustrating a head covering of the cooled insulated headgear device, constructed in accordance with the present invention, with the head covering being constructed of a hard plastic material.

FIG. 3 is a bottom perspective view illustrating the cooled gel insert inserted into the head covering of the cooled insulated headgear device, constructed in accordance with the present invention.

FIG. 4 is a perspective view illustrating the head covering of the cooled insulated headgear device, constructed in accordance with the present invention, with the head covering in the style of a contemporary dare devil type helmet.

FIG. 5 is a perspective view illustrating the head covering of the cooled insulated headgear device, constructed in accordance with the present invention, with the head covering in the style of a cap with an attached hair piece in the back and showing a rechargeable battery on the chin strap.

FIG. 6 is a bottom perspective view illustrating the head covering of the cooled insulated headgear device, constructed in accordance with the present invention, with the head covering in the style of the cap with the attached hair piece in the back and showing the inserted cooled gel insert.

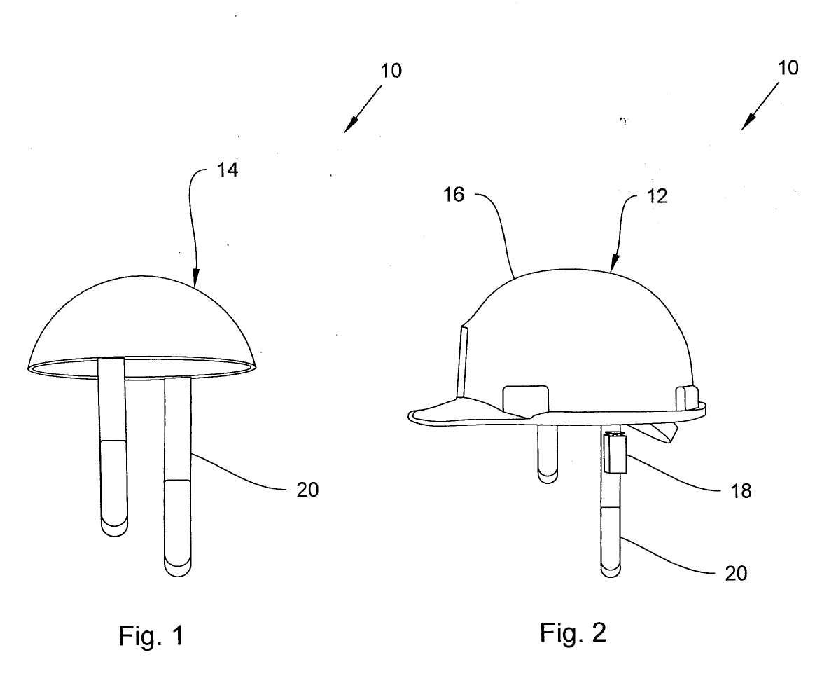

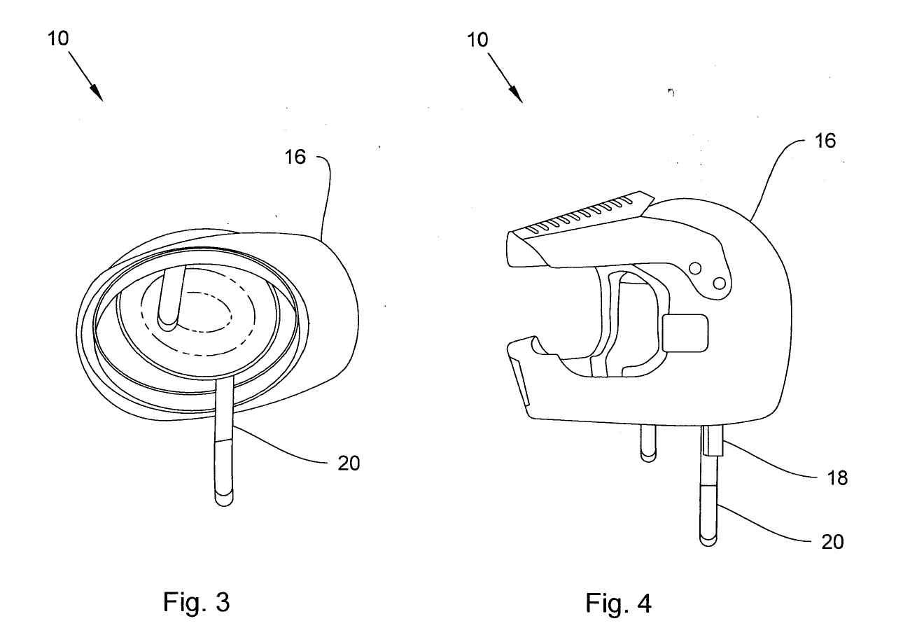

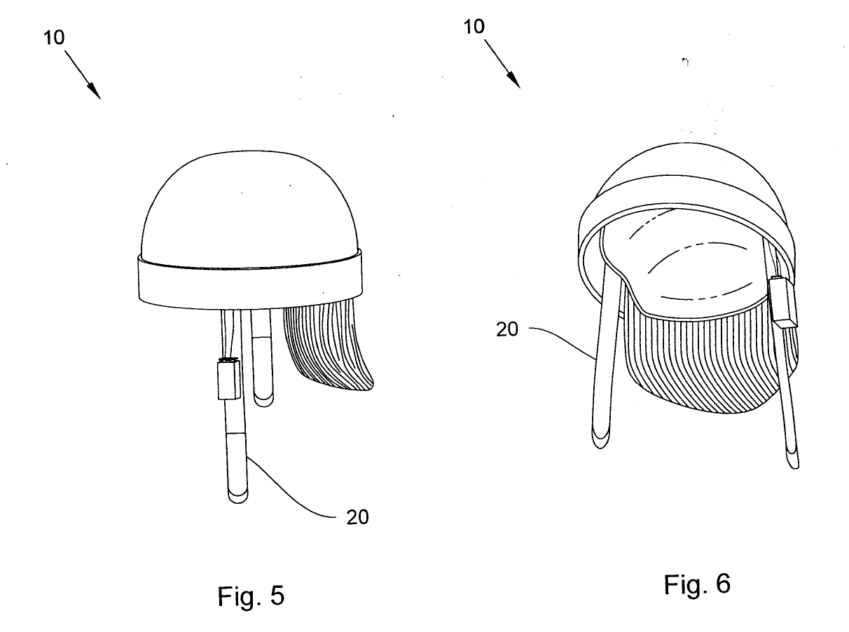

### DETAILED DESCRIPTION OF THE INVENTION

The present invention, hereinafter referred to as the Cooled Insulated Headgear Device, indicated generally at 10, provides cancer patients undergoing chemotherapy with a reliable aid that effectively prevents their hair from falling out due to chemotherapy treatments. The Cooled Insulated Headgear Device 10 is a head covering 12 with a cooled gel insert 14 that keeps the interior of the head covering 12 icy cold or other desired cooled temperature during wear. In a preferred embodiment, the head covering 12 is insulated although having a head covering 12 which is not insulated is within the scope of the present invention.

Preferably, the head covering 12 of the Cooled Insulated Headgear Device 10 of the present invention comprises a tightly fitted cap, hat, or helmet, the interior of which receives the icy cold gel insert 14 that cools the head of the user during wear. The cool therapy of the cooled gel insert 14 functions to reduce blood flow to a wearer's scalp, preventing chemotherapy drugs from reaching and harming hair follicles. In this manner, the chance that hair follicles are damaged as a result of chemotherapy is effectively reduced, enabling a patient wearing the head covering 12 of the Cooled Insulated Headgear Device 10 to maintain their hair during their course of treatment.

The head covering 12 of the Cooled Insulated Headgear Device 10 of the present invention can be a malleable and deformable head covering constructed to encompass the top, sides, and base of a human head, conforming to a scalp and providing a comfortable, customized fit. In a helmet embodiment 16 of the head covering 12, the exterior of the helmet of the Cooled Insulated Headgear Device 10 comprises a hard plastic insulated shell that prevents cold air from escaping the helmet 16 during wear. The icy cold gel insert 14 positioned in the head covering 12 is a cushiony soft gel liner preferably covered in a fine, medical grade plastic and nylon material that transmits the cool temperature to the head and scalp, enabling the wearer to remain comfortable during the process.

The Cooled Insulated Headgear Device 10 of the present invention further includes a hook and loop strap chin fastener 20 can attached to the base of the opening of the helmet embodiment 16 of the head covering 12 or directly to the cool gel insert to tightly secure the helmet to the wearer's head and ensure the helmet 16 remains properly positioned at all times during wear. A rechargeable battery 18 located in the base of the helmet 16 or along the chin strap 20 powers an electrical cooling system which maintains the cool gel insert 14 at a desired cold temperature.

Using the Cooled Insulated Headgear Device 10 of the present invention is simple and straightforward. Prior to undergoing a chemotherapy treatment, a user applies a fully charged Cooled Insulated Headgear Device 10 to their head, making any necessary adjustments to the strap fastener 20 for a secure and comfortable fit. During treatment, the icy cold cooling gel insert 14 lining the interior of the head covering 12 cools the surface of the scalp and hair follicles, restricting the flow of blood containing chemotherapy chemicals to this area. After completing the chemotherapy treatment, the user removes the head covering 12 of the Cooled Insulated Headgear Device 10, exposing their head and scalp to warm ambient temperatures and returning the circulation to this area to normal.

The Cooled Insulated Headgear Device 10 of the present invention prevents toxic chemicals from reaching the scalp and follicles, thus ensuring they remain healthy and strong during cancer treatment. Providing a simple way of preventing hair loss, wearing the Cooled Insulated Headgear Device 10 during treatment results in a patient having improved outlook and positive attitude for a patient. Although intended specifically for wear during chemotherapy treatments, the Cooled Insulated Headgear Device 10 is wearable by individuals who work or spend their leisure time outdoors during hot summer months. Whether a construction worker at a job site in the blazing sun or an avid jogger enjoying a long run on a hot summer day, the Cooled Insulated Headgear Device 10 functions to keep the head, and thus the body, cool and comfortable. Fully rechargeable and durably constructed of high quality materials, the Cooled Insulated Headgear Device 10 will withstand many years of continued use.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claim.

1. A headgear device for inhibiting a user from losing hair, the user having a scalp, the hair growing from hair follicles in the scalp, the headgear device comprising:
a head covering; and
a cooled gel insert receivable within the head covering;
wherein the cooled gel insert cools the scalp and the hair follicles, restricting the flow of blood to the scalp and the hair follicles.

2. The headgear device of claim 1 wherein the head covering is a cap.

3. The headgear device of claim 2 wherein the cap is malleable and deformable.

4. The headgear device of claim 1 wherein the head covering is a hard-sided helmet.

5. The headgear device of claim 4 wherein the helmet is insulated inhibiting cold air from escaping the helmet during wear.

6. The headgear device of claim 1 wherein the head covering encompass the top, sides, and base of the user.

7. The headgear device of claim 1 wherein the cooled gel insert is covered in a fine, medical grade plastic and nylon material that transmits the cool temperature to the scalp of the user.

8. The headgear device of claim 1 and further comprising:
an electrical cooling system for maintaining the cooled gel insert at a desired cooled temperature.

9. The headgear device of claim 8 and further comprising:
a rechargeable battery for powering the electrical cooling system.

10. The headgear device of claim 1 and further comprising:
a chin strap attached to a base of an opening of the head covering.

11. The headgear device of claim 10 and further comprising:
a hook and loop fastener for securing the chin strap.

12. The headgear device of claim 1 wherein the cooled gel insert covers only a portion of an interior surface of the head covering.

13. The headgear device of claim 1 wherein the cooled gel insert completely covers an interior surface of the head covering.

14. A method for inhibiting a user from losing hair, the user having a scalp, the hair growing from hair follicles in the scalp, the method comprising:
providing a head covering;
positioning a cooled gel insert within the head covering;
cooling the scalp and the hair follicles; and
restricting the flow of blood to the scalp and the hair follicles.

15. A headgear device for inhibiting a user from losing hair, the user having a scalp, the hair growing from hair follicles in the scalp, the headgear device comprising:
an insulated, hard-sided head covering;
a cooled gel insert receivable within the head covering;
an electrical cooling system for maintaining the cooled gel insert at a desired cooled temperature;
wherein the insulated head covering inhibits cold air from escaping the helmet during wear; and
wherein the cooled gel insert cools the scalp and the hair follicles, restricting the flow of blood to the scalp and the hair follicles.

16. The headgear device of claim 15 wherein the head covering encompass the top, sides, and base of the user.

17. The headgear device of claim 15 wherein the cooled gel insert is covered in a fine, medical grade plastic and nylon material that transmits the cool temperature to the scalp of the user.

18. The headgear device of claim 15 and further comprising:
a rechargeable battery for powering the electrical cooling system.

19. The headgear device of claim 15 wherein the cooled gel insert covers only a portion of an interior surface of the head covering.

20. The headgear device of claim 15 wherein the cooled gel insert completely covers an interior surface of the head covering.
